(19) United States
(12) Patent Application Publication
(10) Pub. No.: US 2014/0371699 A1
(43) Pub. Date: Dec. 18, 2014

(54) CLOSED OSTOMY BAG SYSTEM

(71) Applicant: Julie Feingold, Alachua, FL (US)

(72) Inventor: Julie Feingold, Alachua, FL (US)

(21) Appl. No.: 13/917,359

(22) Filed: Jun. 13, 2013

(51) Int. Cl. A61F 5/442

(52) U.S. Cl. CPC A61F 5/442
USPC 604/334

(57) ABSTRACT

A system has an ostomy bag and a waste collecting bag. A discharge passage of the ostomy bag can extend into a receiving passage of the collecting bag to receive waste and flushing liquid from the ostomy bag. Slide fasteners connect the passages during the discharging operation. Nubs on the discharge passage engage registration openings in the receiving passage to register the slide fasteners with each other and facilitate their engagement. A vent opening with further slide fastener can be opened to vent gas or to admit flushing liquid into the ostomy bag. A slide fastener extending across the receiving passage of the collecting bag is sealed after the ostomy bag has been clamped and prior to disengaging ostomy bag from the collection bag for sanitary disposal. A hygienic cap is placed over the opening of the ostomy bag to contain any residue contaminants.

### FIELD AND BACKGROUND OF THE INVENTION

The present invention relates generally to the field of post surgical equipment, and in particular to a new and hygienic closed ostomy bag system (COBS).

An ostomy is an opening in the abdominal wall that is made during surgery. In the case of a colostomy, the end of the colon (large intestine) is brought through this opening to form a stoma. An ileostomy is when the last section of the small intestine is opened and brought to the abdominal surface. Where the stoma will be on the abdomen depends on which part of the colon is used to make it. Some ostomies are large, some small, some are on the left side of the abdomen, some are on the right side, others may be in the middle.

Evacuation of digestive products are routed through the stoma, rather than the rectum and anus. Unlike the anus, the stoma has no valve or shut-off muscle. As a result, the ostomy patient is not able to control the passage of stool from the stoma, necessitating the continuous use of an ostomy bag (colostomy or ileostomy) to collect and contain the effluent.

The higher up in the colon the ostomy is made, the shorter the colon. The less time the colon has to absorb water, the softer or more liquid the stool is likely to be. An ostomy further down in the colon, near the rectum, will put out stool that has been in the intestine a longer time. Depending on the effects of illness, medicines, or other forms of treatment, the longer colon can put out a more solid or formed stool. Some people with ostomies find that they are able to pass this stool at certain times of the day with or without the help of irrigation.

Ostomy bags are designed with a tapered, re-closable (utilizing a clip) opening through which the waste is expelled. The ostomy bag is emptied on a regular basis and/or when it fills to a third to half capacity. This occurs at least three times a day. In hospitals, where CDiff may cause diarrhea, it is not uncommon to empty the bag 6 to 10 times a day.

Ostomy bags are emptied into a basin or directly into the commode.

### CURRENT SITUATION/PROBLEM

1. Emptying: Current routine care (while the bag is still attached to the User): The User unfastens the clamp at the mouth of the ostomy bag and positions the mouth over the commode or in a basin. Waste from the ostomy bag is thus emptied through the tapered end into the pan or directly into the commode.

### Problems

a) Emptying fecal waste directly into the commode or basin presents a problem of spills, splashes and odor.

b) In hospitals the spread of HIV, CDiff, MRSA, Hepatitis, etc. via fecal waste is a major concern.

c) If traveling, the User is exposed to unsanitary toilets.

2. Rinsing: After emptying, ostomy bags are rinsed by squirting water up (against gravity) through the mouth at the distal end of the bag. This is the only viable opening available. The mouth is then held closed and the ostomy bag is shaken for the water to wash over the stoma and around in the bag. Once rinsed thoroughly, the mouth is opened and the waste water is emptied out.

### Problems

a) Waste has just been evacuated through the same opening. In order to open mouth so water can be squirted in, users hands now comes in contact with the fecal residue at the mouth of the bag.

b) Holding the mouth open, the user (whose hands have come in contact with fecal residue) now reaches for the irrigation water bottle—contaminating the outside of the bottle.

c) Rinsed residue is evacuated in the same manner as fecal waste. The pan is still in position so the problems of spills and splashes still exist—a field day for the spread of bacteria.

3. Closing: With the same gloves used to evacuate waste, open mouth, reach for squirt bottle to rinse, the user now reaches for the clamp and fastens it over the tapered end of the ostomy bag.

### Problem

The entire area—the outside area of the ostomy bag, the clamp, the squirt bottle, the basin, the hands and surrounding area if any splash has occurred is now a source for spreading bacteria.

4. Cleaning and Storing: If using a pan to empty contents, pan now has to be emptied, cleaned and stored as well as the squirt bottle.

### Problem

Sanitizing and storing equipment for next use.

5. Gas Release: Gas build up is released through the only viable opening at the distal end of the ostomy.

### Problem

Opening the mouth to release gas also invites the probability of leakage of fecal material.

Attempts have been made to solve one or more of these problems.

U.S. Pat. No. 4,387,713 to Calanni discloses a disposable discharge collector for a drainable stoma pouch. The discharge collector is essentially a bag having an opening wide enough to receive the tapered lower portion of the stoma pouch. The bag includes a wiper mechanism for applying pressure to the outside of the stoma pouch, enabling the user to apply pressure to push the matter in the pouch down the tapered lower portion and into bag. The collector includes a slide fastener seal to close the bag but not to connect the colostomy bag to the collecting bag. There is no disclosure in Calanni patent that the disposable discharge collector is attached to the stoma pouch in any way, or is sealed to the pouch to create a closed system. The slide fastener seal of the discharge collector is only used to seal and close the opening so discharge collector can be disposed of without spillage. There is also no disclosure of a vent or port in the top of the bag, or any way to rinse material into the discharge collector.

U.S. Patent Application 2010/0152686 to Ryder et al. discloses a disposable receptacle for receiving bodily waste material from the discharge outlet of a collecting bag. The receptacle has securing means in the form of adhesive strips for attaching the receptacle to collecting bag in a position to receive bodily waste material. The adhesive strips secure the receptacle to the collecting bag and also adhere to each other, thereby forming a leak proof seal. Receptacle is flushable. There is no disclosure in the Ryder Application of a collecting bag having a port or any other means to allow rinsing of the material in the collecting bag into the receptacle. There is also no disclosure of a pressure seal on a bag.

U.S. Patent Application 2008/0065031 to Turner discloses a colostomy bag having a hole for insertion of an air release tube to allow venting of gas from the bag.

U.S. Pat. No. 4,810,250 to Ellenberg et al. discloses a kit for ostomy care including features for rinsing a collection pouch. In the Ellenberg patent, the squeeze bottle connects via a tube to a vent and flush head which passes through the wall of collection pouch to enable flushing the pouch with water squeezed from the bottle.

U.S. Patent Application 2006/0106354 to Vantroostenberghe discloses an ostomy bag with top gas vent and one way valve as well as a collecting tube with spring clip for connecting the outlet passage of the ostomy bag to the collecting tube.

A need remains for a simple and effective system that solves all of the current problems facing the use and cleaning of colostomy and like bags.

### SUMMARY OF THE INVENTION

It is an object of the present invention to provide a closed system for routine emptying and cleaning of an ostomy, colostomy, ileostomy and other like bags (here called ostomy bags for simplicity).

According to the invention, the ostomy bag is provided with a sealable port or vent at the top of the bag, and a plastic seal rim at the mouth of the bag located at the distal end. A disposable waste collection bag is provided with a plastic seal rim at the mouth of the bag. The port seal and seal rims are of the slide fastener type used on sandwich and freezer bags (hereinafter a slide fastener seal). The mouth of the disposable bag is larger than the mouth of the ostomy bag.

In use, the mouth of the disposable bag receives the mouth of the ostomy bag, enabling the respective bag seals to register and be pinched together, forming a liquid-impermeable seal. The ostomy bag is then unclamped allowing passage of material to flow downward from the ostomy bag into the disposable bag. To rinse the ostomy bag, the top port (or vent) is opened and water is squirted through the port, washing material down into the disposable bag. Due to the liquid-impervious seal between the ostomy bag and disposable bag, the ostomy bag can be rinsed without spills, splashes and odor. After rinsing the ostomy bag, it is clamped as usual, the disposable bag is removed and discarded or flushed.

Advantageously the disposable bag also includes a second slide seal for closing the inlet passage of the disposable waste collection bag after it has received waste from the ostomy bag. Before detaching the waste collection bag, the inner seal is pinched shut to contain the waste and thus facilitate its disposal without the danger of leakage or spills.

Projecting nubs or posts are also provided on opposite sides of the outlet seal of the ostomy bag that engage respective holes on the inlet seal of the collection bag. This feature makes it easier for the user to center the parts of the slide fasteners on the outer surface of the outlet passage of the ostomy bag, with the mating parts of the slide fasteners on the inner surface of the inlet passage of the collection bag. Once aligned, the slide fasteners of the ostomy bag and collection bag are pinched together and thus securely connecting the ostomy bag outlet to the collection bag inlet. After this is done, the clip or clamp of known design that keeps the outlet passage of the ostomy bag closed during use, is released to allow the waste to empty from the ostomy bag to the collection bag.

The top vent that can be used to release gas from the ostomy bag between emptying operations, can then be opened to allow access for a flushing bottle nozzle to further clear the ostomy bag.

The new Closed Ostomy Bag System (COBS) of the invention addresses the problems of the prior art as outlined above. It provides for minimal redesign of the current ostomy bag. It proves the user the option to use the closed system or empty as usual, with the one improvement of a top-side port for rinsing and releasing gas. It provides a Collection Bag (CB) which can be disposable or flushable depending on the material specified by the manufacturer and it provides for a plastic interlocking seal to accomplish a closed system whereby the transfer of waste product from the ostomy bag to the CB is possible.

Interlocking seal can be used with featured CB or larger bag for irrigation. It is strong enough to hold in place an elongated outlet (as used in fecal management systems) that can be attached to a bedside bag in the case of diarrhea.

The inventive redesign provides two important changes to the current ostomy bag: a plastic sealable port at the top of the bag and a plastic seal rim at the mouth of the bag.

In addition, the invention provides for a secondary Collection Bag (CB) with a plastic seal rim at the mouth of the bag that interlocks with the seal of the ostomy bag providing a secure passageway for the discharge of waste material from the ostomy bag into the CB.

The foremost consideration of the invention is to utilize existing art as far as possible to provide for a viable solution to the current situation/problems with minimal start up expense to manufactures and the opportunity for manufacturers to increase their bottom line.

Port or Vent: a plastic seal (as currently used to seal sandwich, freezer bags) opening located at the top-side of the ostomy bag for easy cleaning. The position at the top of the ostomy bag utilizes gravity to wash the cleaning solution (water) flow downward and out the mouth of the ostomy bag. Port Secondary Use: provides for a vent to relieve “gas” build up.

The plastic Seal at the mouth of the ostomy bag to interlock with the seal at the mouth of the CB comprising a COBS. The ridge side of the seal faces the outside of the ostomy bag, leaving the inside of the mouth smooth for the flow of effluent. Once interlocked, waste flows from the ostomy bag into the CB.

Collection Bag (CB): attaches to the mouth (distal end) of the ostomy bag via a plastic seal. The groove side of the seal in on the inside of the CB. The mouth of the CB is slightly larger than the mouth of the ostomy bag so it can fit over the mouth of the ostomy bag. Once aligned, the bags can be “snapped” in place. The COBS assures a more sanitary environment and facilitates routine bag emptying and cleaning while in its operable position on the User.

Interlocking Plastic Seal: has a groove and ridge. In order for them to interlock, they must be in alignment. Up to this point, this type of plastic seal has been used to close and contain—alignment was not a problem as the seal was manufactured in a fixed position so the groove and ridge could easily be “snapped” together. This innovation calls for the interlocking of two art forms to form a conduit for the flow of material from one art form to the other utilizing the seal previously designed to contain. For added strength, a double seal (as used for freezer bags) is used in connecting the ostomy bag to the CB.

Without a fixed position, the user may experience difficulty (visual, tactile) in aligning the groove and ridge. To facilitate alignment, a “nub” is fashioned on either side of the mouth of the ostomy seal between the two ridges. A corresponding small hole is inserted at the ends of the CB seal so that when nubs are inserted into the corresponding holes, the ridges and grooves are aligned and the seal can be easily “snapped” closed.

Cap for Ostomy Opening: has a hole on either side to fit over nubs of ostomy seal.

The Closed Ostomy Bag System (COBS) provides a Collection Bag (CB) that is secured to the mouth (bottom) of the ostomy bag by a plastic seal (like used in sandwich, freezer bags, etc.). Once attached, the waste empties into the CB. There is no danger of spills and splashes, and odor is kept at a minimum. The spread of disease via exposure to fecal waste is kept at a minimum. The traveler is not exposed to unsanitary public toilets. Can empty bag in private area other than bathroom.

A single plastic sealed “port” or manually closable vent opening is provided at the top of the ostomy bag for rinsing. When accessed, water is squirted through the “port” and washes down into the CB. This provides easy rinsing that flows with gravity down over the stoma. The squirt bottle never comes in contact with fecal residue. Once cleaned, the ostomy bag is clamped as usual and the CB is sealed via a single plastic seal located directly under the double seal. The double seal is then released and the CB removed. An antiseptic swab is recommended to wipe inside the rim to remove any residue. The CB can now be disposed of. No cleaning or storage necessary.

Secondary Use: The “port” may also be used to rid the ostomy bag of gas build up. Currently, if the user has a buildup of “gas”, s/he relieves it by unclamping the mouth of the ostomy bag. This poses a problem: if the bag contains any effluent—waste as well as gas is released and a cleanup is necessary. The top port alleviates this concern. It is recommended that the mouth of the ostomy bag be wiped with an antiseptic swapstick and capped to prevent any contamination.

A double seal (as used for freezer bags) is used in connecting the ostomy bag to the CB. The plastic seal (ridge side) is on the outside—around the mouth of the ostomy bag. The seal's counterpart is on the inside mouth of the CB. To connect, the user aligns the ridge and groove by slipping the nubs on either side of the ostomy bag into the corresponding holes in the CB. The mouth of the CB is larger than the mouth of the ostomy bag so it can fit over the ostomy bag seal.

Once in place, the user pinches together the interlocking pieces of the plastic seal. Once sealed, the ostomy bag is unclamped and the waste allowed to flow from the ostomy bag into the CB without the user ever coming into contact with fecal waste. It is not necessary that the seal between the ostomy bag and the CB be water tight because user empties ostomy when it is one third to half full. The CB is large enough to accompany effluent without filling. Once emptied, the user accesses the sealed top port, squirts water which runs downward rinsing out the bag, and empties into the CB. The ostomy bag is then clamped. There is a single, secondary seal just below the double seal at the mouth of the CB designed to contain the waste contents. It is locked prior to disengaging the 2 bags. Once the CB is released from the ostomy bag, an antibacterial swab stick is recommended to wipe the inside mouth and rim of the ostomy bag. A snap on cap for the tip of the ostomy mouth is provided to assure any residue from the transfer of waste is contained.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which a preferred embodiment of the invention is illustrated.

### BRIEF DESCRIPTION OF THE DRAWINGS

In the drawings:

FIG. 1 is an exploded view of a ostomy bag system of the invention with part of the receiving passage of a collecting bag of the system cut away to show the slide fasteners on the inside surfaces thereof;

FIG. 2 is a sectional view taken along line 2-2 of FIG. 1;

FIG. 3 is a sectional view taken along line 3-3 of FIG. 1;

FIG. 4 is a detail view taken from area 4 of FIG. 1 plus a partial view of a colostomy bag discharge passage of the invention;

FIG. 5 is a sectional view of the slide fasteners in the passages of the ostomy bag and collecting bag of FIG. 1, when they have been registered or positioned to be connected to each other; and

FIG. 6 is a sectional view of the slide fasteners in the passages of a ostomy bag and collecting bag of a further embodiment of the invention.

### DESCRIPTION OF THE PREFERRED EMBODIMENTS

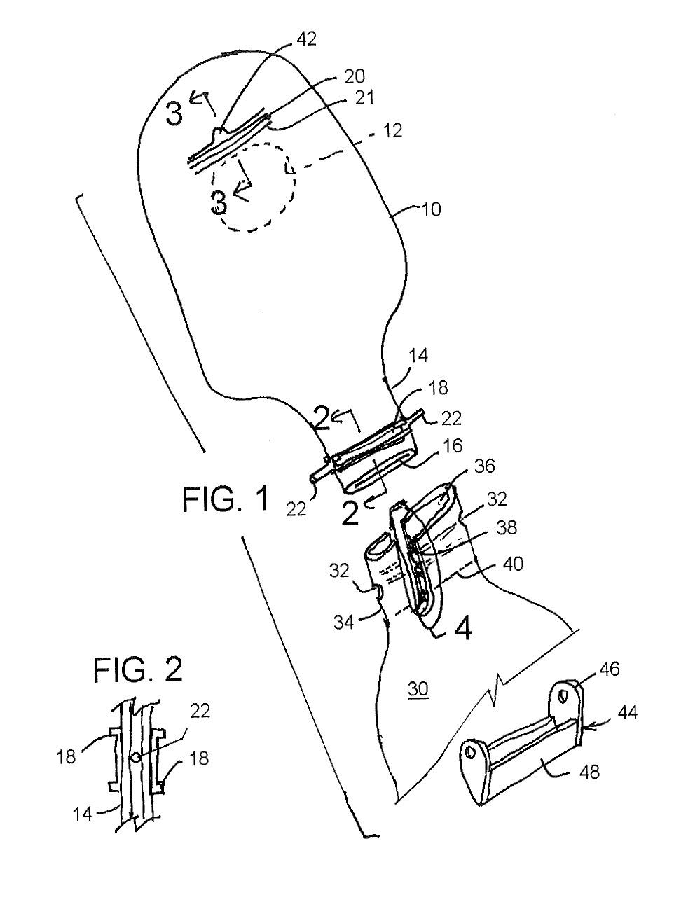

Referring now to the drawings, in which like reference numerals are used to refer to the same or similar elements, FIG. 1 shows and example of an ostomy bag system comprising a colostomy bag 10 having an upper end toward the top of the page, a lower end and a first surface for laying adjacent a patient with an colostomy stoma. The bag has an colostomy opening 12 of known design in its first surface near the upper end of the colostomy bag for communicating with the stoma to receive waste from the patient.

The colostomy bag includes a second surface facing the reader that is opposite from the first surface and an elongated discharge passage 14 at the lower end of the colostomy bag that is of a first transverse dimension that is narrower than the remainder of the bag. The discharge passage 14 has a discharge opening 16 at its bottom end for discharging waste from the colostomy bag. During use of the colostomy bag, the passage 14 is closed by a clip or clamp (not shown) of known design and the bag collects waste from the patient.

A first slide fastener portion 18, e.g. one or more elongated male slide fastener parts, is glued or heat sealed to each of the first and second outer surfaces of the colostomy bag at the discharge passage. Each is for connecting to a mating (e.g. female) second slide fastener portion to be explained later.

All of the slide fasteners and slide fastener portions used by the invention are of the known zip lock type having one elongated male portion or ridge that can be firmly seated in a water tight manner into an elongated female valley or trough of the other fastener portion.

At least one, but preferably two opposite nubs 22 project to the sides from the discharge passage 14, each for engaging a registration opening to be explained later for positioning and registering the first slide fastener portions 18 with the second, mating slide fastener portions.

A vent opening 20 in the second surface of the colostomy bag 10 at or above the ostomy opening 12 is also present and has a third slide fastener 21 for closing the vent opening 20. The slide fastener 21 is normally closed in use but can be manually opened for opening the vent opening 20 to vent gas from the colostomy bag 10 or to admit flushing liquid into the colostomy bag as part of the bag emptying operation. The colostomy bag system also includes a grasping tab 42 shown in FIG. 1, projecting from an outer slide fastener portion of the third slide fastener 21 for facilitating manual opening of the third slide fastener.

The system also includes a waste collecting bag 30 having a receiving passage 34 with a waste receiving opening 36. The receiving passage 34 is slightly larger in transverse dimension than the first dimension of the colostomy bag discharge passage 14, so that the discharge passage 14 can extend into the receiving passage 34 when waste is to be discharged from the colostomy bag 10 to the collecting bag 30.

A second slide fastener portion 38, e.g. one or more female slide fastener portions, are on each opposite inside surface of the receiving passage 34, each for mating with one of the first slide fasteners 18 for connecting the colostomy and collecting bags to each other at their respective passages to facilitate discharging of waste from the colostomy bag to the collecting bag.

A pair of each of the slide fastener portions 18 and 38 are preferred for strength and redundancy to avoid leakage and also so that the weight of the filling collecting bag will not disconnect the bags from each other. Any combination of male and female slide fastener portions can be used of the mating fastener portion pairs 18 and 38. In one embodiment shown in FIG. 6, the opposite fastener portions 38 can be of opposite mating type so they can mate either with the fastener portion pairs 18 of bag 10, or with each other to close the collecting bag 30 for disposal.

The receiving passage 34 including at least one but better two opposite registration openings 32, each to be engaged by one of the nubs 22 to facilitate lining up and registering the first and second slide fastener portions with each other.

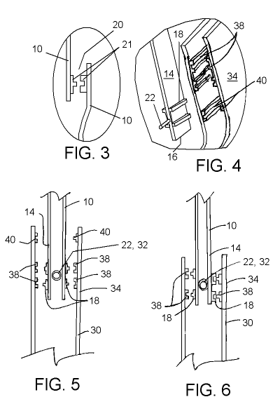

As shown in FIGS. 1 and 4, a fourth slide fastener 40 extends across the receiving passage 34 and on inside surfaces of the receiving passage 34 for closing the receiving passage after it has received waste and any flushing liquid from the colostomy bag 10 so that the collecting bag 30 can be closed for sanitary disposal.

In this preferred embodiment the fourth slide fastener 40 is downstream of the second slide fastener 38 and is also downstream of the end of passage 14 and its opening 16. In this way the collection bag 30 can be sealed closed before it is disengaged from the colostomy bag 10 by disengaging seals 18 from seals 38. In other embodiments it can be positioned upstream but this would have to be sealed after disengaging the two bags. The alternated embodiments of FIGS. 5 and 6 require that the collection bag 30 be sealed after it is detached from the colostomy bag 10.

The embodiment shown in FIG. 5, only requires the mating fastener portion pairs 18 and 38 to mate with each other and not for the opposite pairs 38 of the collecting bag to mate with each other as in the embodiment of FIG. 6.

The fourth slide fastener 40 is either downstream of the second slide fastener 38 portions as shown in FIG. 1, or upstream of the second slide fastener 38 portions as shown in FIG. 5. As mentioned, the colostomy bag system should have first and second slide fasteners 18 and 38 that are double slide fasteners as shown.

In the embodiment of FIG. 6, one of the colostomy bag fastener portions 18 on one side of the discharge passage are a pair of male fastener portions while the other portions 18 are a pair of female fastener portions. Complementary fastener portions 38 are provided on the elongated receiving passage of the collecting bag 30. Alternatively, one of the pair 18 may be male and the other female, and one of the pair 38 may be female and the other male.

By selecting the proper combination of male and female slide fastener portions, the opposite inside surface-mounted fastener portions 38 can mate with each other to seal the collecting bag closed for disposal and therefore not require a separated fourth slide fastener 40 as needed in the embodiments of FIGS. 1 and 5.

The colostomy bag system preferably has first and second slide fastener portions 18 and 38 that are double slide fastener portions, there being a pair of nubs 22 projecting from opposite sides of the discharge passage 14 adjacent the first slide fastener portions 18, the receiving passage 34 having a registration opening 32 for each nub, adjacent the second slide fastener portions 38. Since the materials of the bags and fasteners are transparent, this registration system is important to allow the person emptying the colostomy bag 10 to use both sight and feel to line up the pairs of fasteners 18 and 38 with each other and then press the lined up fasteners against each other to close the seals. After that is done, the clip of clamp that has kept the passage 14 closed until then, can be opened to allow the waste to flow by gravity out of the colostomy bag 10 and into the collecting bag 30.

The colostomy bag system also includes a cap 44 in FIG. 1, having ears 46 with openings for receiving the nubs 22 and a trough 48 between the ears for receiving the discharge opening 16 and for restraining leakage from the discharge opening after the bags 10 and 30 are disconnected from each other.

While specific embodiments of the invention have been shown and described in detail to illustrate the application of the principles of the invention, it will be understood that the invention may be embodied otherwise without departing from such principles.

What is claimed is:

1. An ostomy bag system comprising:
an ostomy bag (10) having an upper end, a lower end, and a first surface for laying adjacent a patient with an ostomy stoma, an ostomy opening (12) in the first surface near the upper end of the ostomy bag for communicating with the stoma to receive waste from the patient, the ostomy bag including a second surface that is opposite from the first surface and a discharge passage (14) at the lower end of the ostomy bag, having a first dimension, the discharge passage having a discharge opening (16) for discharging waste from the ostomy bag;
a first slide fastener portion (18) on each of the first and second surfaces of the ostomy bag at the discharge passage (14), each for connecting to a mating second slide fastener portion;
at least one nub (22) projecting from the discharge passage (14) for engaging a registration opening to position the first slide fastener portions (18) with the second slide fasteners;
a vent opening (20) in the second surface of the ostomy bag at or above the ostomy opening (12);
third slide fastener (21) for closing the vent opening and for being manually opened for opening the vent opening to vent gas from the ostomy bag or to admit flushing liquid into the ostomy bag;
a waste collecting bag (30) having a receiving passage (34) with a waste receiving opening (36), the receiving passage being larger than the first dimension so that the discharge passage (14) extends into the receiving passage (34) when waste is to be discharged from the ostomy bag to the collecting bag;
a second slide fastener portion (38) on each opposite inside surface of the receiving passage (34), each for mating with one of the first slide fastener portions (18) for connecting the ostomy and collecting bags to each other at their respective passages to facilitate discharging of waste from the ostomy bag to the collecting bag;
the receiving passage (34) including at least one registration opening (32) for being engaged by the nub (22) to register the first and second slide fastener portions with each other; and
a fourth slide fastener (40) extending across the receiving passage (34) and on inside surfaces of the receiving passage for closing the receiving passage after it has received waste and any flushing liquid from the ostomy bag so that the collecting bag can be closed for sanitary disposal.

2. The ostomy bag system according to claim 1, wherein the fourth slide fastener (40) is separate from the second slide fastener portions and is downstream of the second slide fastener (38) portions and is downstream of the discharge opening (16) when the first and second slide fastener portions are engages with each other so that the fourth slide fastener (40) can be closed before the first and second slide fastener portions are disengaged to reduce possible leakage.

3. The ostomy bag system according to claim 1, wherein the fourth slide fastener (40) is separate from the a second slide fastener portions and is upstream of the second slide fastener (38) portions.

4. The ostomy bag system according to claim 1, wherein each of the first and second slide fasteners (18, 38) are double slide fasteners.

5. The ostomy bag system according to claim 1, wherein each of the first and second slide fastener portions (18, 38) are double slide fastener portions, there being a pair of nubs (22) projecting from opposite sides of the discharge passage (14) adjacent the first slide fastener portions (18), the receiving passage (34) having a registration opening (32) for each nub, adjacent the second slide fastener portions (38).

6. The ostomy bag system according to claim 1, including a grasping tab (42) projecting from an outer slide fastener portion of the third slide fastener (21) for facilitating manual opening of the third slide fastener.

7. The ostomy bag system according to claim 1, including a pair of nubs (22) projecting from opposite sides of the discharge passage (14) adjacent the first slide fastener portions (18), the receiving passage (34) having a registration opening (32) for each nub adjacent the second slide fastener portions (38), and a cap (44) having ears (46) with openings for receiving the nubs and a trough (48) between the ears for receiving the discharge opening (16) and for containing any residue contaminants from the discharge opening.

8. An ostomy bag system comprising:
an ostomy bag with an ostomy opening for communicating with the stoma of a patient to receive waste from the patient, the ostomy bag including a discharge passage having a discharge opening for discharging waste from the ostomy bag;
a first slide fastener portion on each opposite outside surface of the discharge passage, each for connecting to a mating second slide fastener portion;
a vent opening in the ostomy bag at or above the ostomy opening, and a slide fastener for closing the vent opening and for being manually opened for opening the vent opening to vent gas and to admit flushing liquid into the ostomy bag;
a waste collecting bag having a receiving passage with a waste receiving opening, the receiving passage being larger than the discharge passage so that the discharge passage extends into the receiving passage when waste is to be discharged from the ostomy bag to the collecting bag; and
a second slide fastener portion on each opposite inside surface of the receiving passage for mating with one of the first slide fasteners for connecting the ostomy and collecting bags to each other at their respective passages to facilitate discharging of waste from the ostomy bag to the collecting bag.

9. The ostomy bag system according to claim 8, wherein the one of the first and second slide fastener portions is a male slide fastener and another of the first and second slide fastener portions is a female slide fastener so that after waste has been collected the second slide fasteners can be mated to each other to close the collecting bag for disposal.

10. The ostomy bag system according to claim 8, wherein each of the first and second slide fastener portions are double slide fastener portions, there being a pair of nubs projecting from opposite sides of the discharge passage adjacent the first slide fastener portions, the receiving passage having a registration opening for each nub, adjacent the second slide fastener portions.

11. The ostomy bag system according to claim 8, including a grasping tab projecting from an outer slide fastener portion of the further slide fastener for facilitating opening of the third slide fastener.

12. The colostomy bag system according to claim 8, including a pair of nubs projecting from opposite sides of the discharge passage adjacent the first slide fastener portions, the receiving passage having a registration opening for each nub adjacent the second slide fastener portions, and a cap having ears with openings for receiving the nubs and a trough between the ears for receiving the discharge opening and for containing any residue contaminant from the discharge opening.

13. The ostomy bag system according to claim 8, including a grasping tab projecting from an outer slide fastener portion of the further slide fastener for facilitating opening of the third slide fastener, and a pair of nubs projecting from opposite sides of the discharge passage adjacent the first slide fastener portions, the receiving passage having a registration opening for each nub adjacent the second slide fastener portions, and a cap having ears with openings for receiving the nubs and a trough between the ears for receiving the discharge opening and for containing any residue contaminant from the discharge opening.

14. The ostomy bag system according to claim 8, wherein each of the first and second slide fastener portions are double slide fastener portions, there being a pair of nubs projecting from opposite sides of the discharge passage adjacent the first slide fastener portions, the receiving passage having a registration opening for each nub, adjacent the second slide fastener portions, the system including a grasping tab projecting from an outer slide fastener portion of the further slide fastener for facilitating opening of the third slide fastener.

15. The ostomy bag system according to claim 8, including a fourth slide fastener that is downstream of the second slide fastener portions and is downstream of the discharge opening when the first and second slide fastener portions are engages with each other so that the fourth slide fastener can be closed before the first and second slide fastener portions are disengaged to reduce possible leakage.
